(19) United States
(12) Patent Application Publication
(10) Pub. No.: US 2006/0063753 A1
(43) Pub. Date: Mar. 23, 2006

(54) Use of nefopam for the treatment of nausea or emesis

(76) Inventors: Robin Mark Bannister, ESSEX CB10 1XL (GB); Michael Lyne, Essex (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/517,881

(22) PCT Filed: Jun. 17, 2003
(86) PCT No.: PCT/GB03/02586

§ 371 Date: Jun. 29, 2005

(30) Foreign Application Priority Data
Jun. 17, 2002 (GB) 0213869.1

(51) Int. Cl. A61K 31/553; A61K 31/573; A61K 31/5415; A61K 31/44

(52) U.S. Cl. 514/211.09; 514/171; 514/317; 514/304; 514/225.8

(57) ABSTRACT

The invention relates to the use of nefopam for the manufacture of a medicament for the treatment of nausea, dizziness, blurred vision and emesis.

### FIELD OF THE INVENTION

This invention relates to the use of a known compound in the treatment of emesis and related conditions.

### BACKGROUND OF THE INVENTION

Nefopam is a centrally acting non-narcotic analgesic not structurally related to other analgesics. Nefopam has been shown to induce antinociception in animal models of pain and in humans (reviewed in Heel et al., Drugs 19(4): 249-67, 1980). However, nefopam is not active in the mouse tail-flick test, the hot plate test or the Randall-Selitto pressure test in rats (Conway and Mitchell, Arch. Int. Pharmacodyn. Ther. 226(1): 156-71, 1977), suggesting that its analgesic mechanism is not opiate-like or anti-inflammatory in nature. Nefopam's antinociception is not blocked by nalaxone, further suggesting that its analgesic action is not through opiate receptors.

In vitro and in vivo studies with nefopam enantiomers have shown that (+)-nefopam has more potent analgesic and dopamine, norepinephrine and serotonin-uptake inhibitory properties than (−)-nefopam, with the order of potency given as (+)-nefopam > (±)-nefopam > (−)-nefopam (Fasmer et al., J. Pharm. Pharmacol. 42(6): 437-8, 1987; Rosland and Hole, J. Pharm. Pharmacol. 42(6): 437-8, 1990; Mather et al., Chirality 12(3): 153-9, 2000). Mather et al. (2000) conclude that “ . . . there is currently no compelling rationale tojustify administering or monitoring individual enantiomers [of nefopam]”.

Nefopam has also been shown to be opiate-sparing when given with morphine in trials of patient-controlled analgesia (Mimoz et al., Anaesthesia 56(6): 520-5, 2001).

Conventional release preparations of nefopam have been commercially available for many years, for use in treating moderate to severe pain. However, the short elimination half-life of nefopam (four hours) means that it is difficult to maintain analgesic efficacy over the normal dosing period (three times daily). Dose escalation of nefopam brings about an increase in the frequency of adverse drug reactions associated with the analgesic, and adverse effects on pulse and blood pressure have been observed following parenteral delivery of therapeutic doses of nefopam (Heel et al., 1980). Chronotropic and ionotropic effects on the heart are not present when nefopam is administered orally (Bhatt et al., Br. J. Clin. Pharmacol. 11(2): 209-11, 1981).

Nausea and vomiting are side-effects of the use of many drugs, including those administered for the treatment of pain.

### SUMMARY OF THE INVENTION

According to the present invention, emesis or a related condition is treated by the use of nefopam. Given nefopam's side-effect profile, it was surprising to find that racemic nefopam and its enantiomers were able to prevent or diminish emesis caused by administration of opioid and other recognised proemetic agents.

### DESCRIPTION OF PREFERRED EMBODIMENTS

As used herein, “nefopam” refers to a compound of formula I 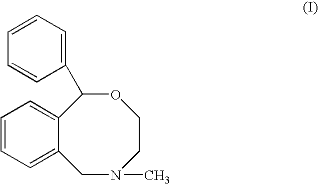
and salts, e.g. the hydrochloride, metabolites and prodrugs thereof, as well as the (+) and (−) enantiomers which are as far as possible optically pure. (+)-Nefopam may be preferrred, for reduced side-effects caused by interaction.

According to the invention, nefopam is used to treat nausea, dizziness, blurred vision or emesis, including, but not limited to, acute, delayed, post-operative, last-phase and anticipatory emesis. This condition may be induced by, for example, chemotherapy, radiation, toxins, pregnancy, alcohol withdrawal, nicotine withdrawal, drug withdrawal, vestibular disorder, motion, post-operative sickness, surgery, gastrointestinal obstruction, reduced gastrointestinal motility, dysmenorrhoea, visceral pain, migraine, increased intracranial pressure, decreased intracranial pressure, depression or opioid analgesics. In addition, nefopam may be used to treat emesis caused by certain drugs such as antidepressants (examples including amitriptyline, imipramine, desipramine, venlafaxine, citalopram, trazadone, paroxetine, nefazodone, fluoxetine and (S)-citalopram), anticonvulsants (examples including lamotrigine, gabapentin and carbamezepine), antipsychotics (examples including clozapine, chlorpromazine, fluphenazine, haloperidol and loxapine), anxiolytics (examples including buspirone and lorazepam), anti-Parkinson's agents (examples including apomorphine, pergolide, levodopa, dopamine, naxagolide, bromocriptine and amantadine), CNS stimulants (examples including dexamphetamine and methylphenidate), opioids (examples including morphine, fentanyl, buprenorphine, codeine, methadone, oxycodone, phenacozine and diamorphine), and anticancer agents (examples including cisplatin, aldesleukin, altretamine, carboplatin, carmustine, cyclophosphamide, cytarbine, decarbazine, dactinomycin, daunorubicin, docetaxel, doxorubicin, epirubicin, fluorouracil, idarubicin, ifosfamide, irotecan, lomustine, mechlorethamine, melphalan, methotrexate, mitoxantrone, pentostatin, procarbazine and streptozocin).

Nefopam may be used according to the invention when the patient is also being given another anti-emetic agent. Such agents include phenothiazines, 5HT3 receptor antagonists, dopamine antagonists, anticholinergic agents, anti-histamines, histamine analogues, cannabinoids, corticosteroids, GABA receptor antagonists, NK1 receptor antagonists, and α₂ and α₃ adrenoceptor antagonists.

Specific examples of these types of compounds are cyclizine, dolasetron, granisetron, ondansetron, tropisetron, nabilone, scopolenine, cinnerizine, promethazine, betahistine, dexamethasome, methylprednisolone, metoclopramide, chlorpromazine, perphenazine, prochlorperazine, thiethylperazine, droperidol, domperidone and haloperidol..

Any suitable route of administration can be used. For example, any of oral, topical, ocular, rectal, vaginal, inhalation and intranasal delivery routes may be suitable. The dose of the active agent will depend on the nature and degree of the condition, the age and condition of the patient, and other factors known to those skilled in the art. A typical dosage is 10-100 mg given one to three times per day.

The evidence upon which this invention is based follows.

Study

Male ferrets (0.9- 1.7 kg) obtained from Leeds University were housed in pairs at 22±1° C. and had free access to food (SDS Diet ‘C’ (E), Special Diet Services, UK) and water. They were housed under artificial lighting with lights on between 07:00 and 21:00 hours. For experimental use, animals were removed from their holding cages and placed individually into observation cages. The animals were allowed free access to water and food. The animals were divided into separate groups of 4 animals per group.

Animals were frequently observed throughout the experiments by a trained technician to ensure that the animals remained in good health. In addition, animal behaviour was video recorded for subsequent analysis of emesis (see Rudd et al., 1994). Emesis was characterized by rhythmic abdominal contractions which were either associated with the oral expulsion of solid or liquid material from the gastrointestinal tract (i.e. vomiting) or not associated with the passage of material (i.e. retching movements). The number of highly distinctive abdominal contractions was counted.

(+)-Nefopam was dissolved in saline and administered in a volume of 1 ml/kg. Normal saline was used as the control vehicle injection. Cisplatin (Cisplatin Injection Sterile Concentrate 50 mgl 50 ml; Onco-Tain: Faulding Pharmaceuticals PLC. Queensway, Royal Leamington Spa, Warwickshire, CV31 3RW,UK) was administered in a volume of 5 ml/kg i.p.

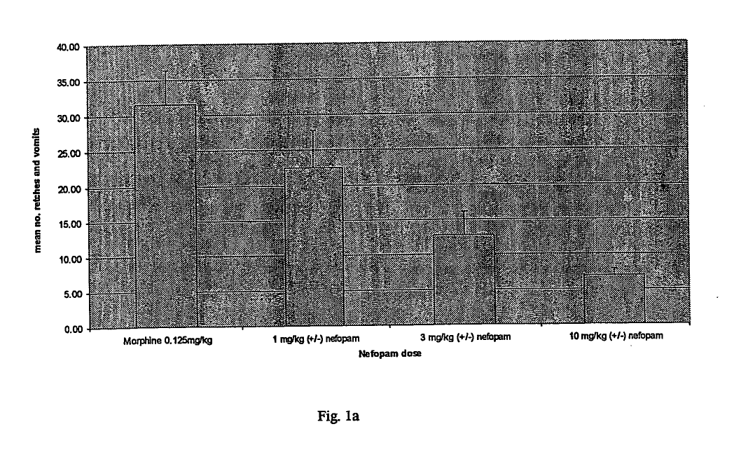

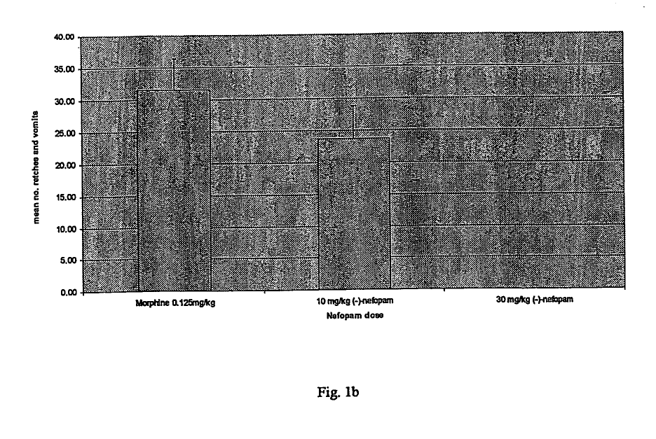

Ferrets (n=4) were pre-dosed intraperitonealy with either racemic nefopam (1, 3 and 10 mg/kg i.p.—FIG. 1a), (−)-nefopam (10 and 30 mg/kg—FIG. 1b) or (+)-nefopam (0.3, 1 and 3 mg/kg—FIG. 1c) one hour prior to being given an emetic dose of morphine (0.125 mg/kg s.c.). Observations were recorded over a 4 hr period post-morphine dosing and scored for incidences of retching and vomiting. Results are shown in FIG. 1.

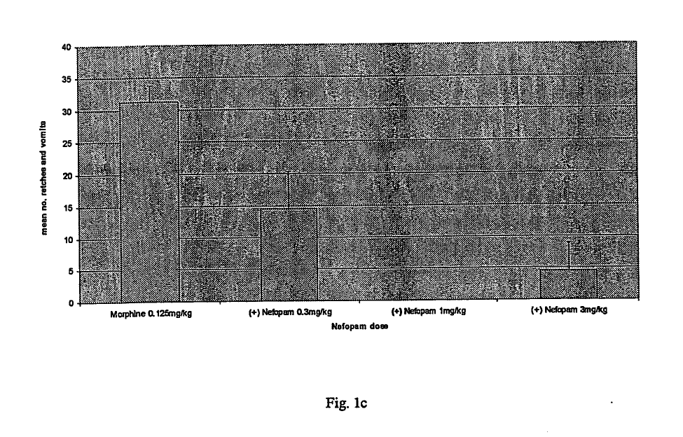

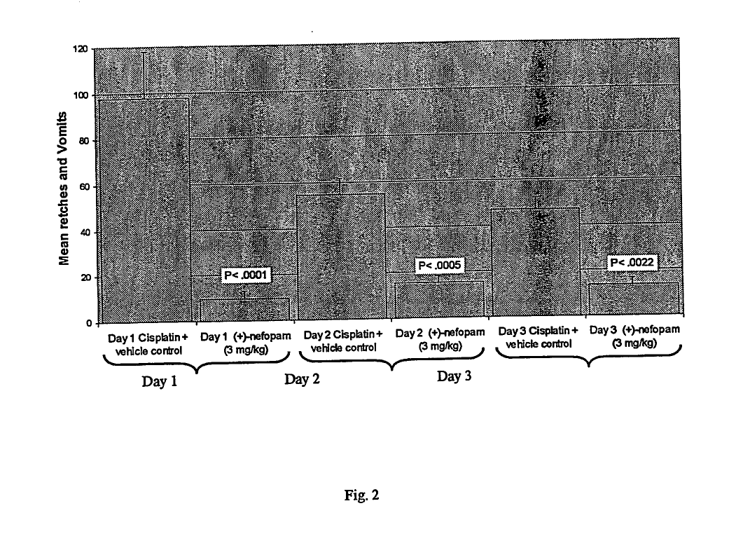

(+)-Nefopam (3 mg/kg) was administered to ferrets (n=4) intraperitonealy three times daily (q8h) starting one day before cisplatin administration (5 mg/kg i.p.) and continuing for three days after cisplatin administration. Observations were recorded over the 72 hr period post-cisplatin dosing and scored for incidences of retching and vomiting. Results are shown in FIG. 2.

1. A method for treating a condition selected from the group consisting of nausea, dizziness, blurred vision and emesis, wherein said method comprises administering, to a patient in need of such treatment, an effective amount of nefopam.

2. The method, according to claim 1, wherein the condition is acute, delayed, postoperative, late-phase or anticipatory emesis.

3. The method, according to claim 1, wherein the condition is associated with dysmenorrhoea, migraine, cancer or other pain condition.

4. The method, according to claim 1, wherein the condition is induced by one or more of radiation, toxins, pregnancy, alcohol withdrawal, nicotine withdrawal, drug withdrawal, vestibular disorder, motion, post-operative sickness, surgery, gastrointestinal obstruction, reduced gastrointestinal mobility, visceral pain or increased or decreased intracranial pressure.

5. The method, according to claim 1, wherein the condition is drug-induced.

6. The method, according to claim 5, wherein the condition is induced by chemotherapy.

7. The method, according to claim 5 wherein the condition is induced by an opioid analgesic.

8. The method, according to claim 1, wherein the patient is also administered another agent that has anti-emetic properties.

9. The method, according to claim 8, wherein said agent is selected from the group consisting of phenothiazines, 5HT3 receptor antagonists, dopamine antagonists, anticholinergic agents, anti-histamines, histamine analogues, cannabinoids, corticosteroids, GABA receptor antagonists, NK1 receptor antagonists, and α₂ and α₃ adrenoceptor antagonists.

10. The method, according to claim 8, wherein said agent is selected from the group consisting of cyclizine, dolasetron, granisetron, andansetron, tropisetron, nabilone, scopolenine, cinnerizine, promethazine, betahistine, dexamethasome, methylpredrisolone, metoclopramide, chlorpromazine, perphenazine, prochlorperazine, thiethylperazine, droperidol, domperidone and haloperidol.
